(19) United States
(12) Patent Application Publication
(10) Pub. No.: US 2018/0296707 A1
(43) Pub. Date: Oct. 18, 2018

(54) PHARMACEUTICAL FORMULATION AND METHOD OF PREPARING THE SAME

(71) Applicant: SeeCure Taiwan Co., Ltd., Kaohsiung City (TW)

(72) Inventors: Ning Tsao, Kaohsiung City (TW); Tsung-Tien Kuo, Kaohsiung City (TW); David J. Yang, Sugar Land, TX (US)

(73) Assignee: SeeCure Taiwan Co., Ltd., Kaohsiung City (TW)

(21) Appl. No.: 15/486,297

(22) Filed: Apr. 12, 2017

(51) Int. Cl. A61K 51/08; A61K 51/06; A61K 9/00

(52) U.S. Cl. CPC A61K 51/088; A61K 9/0019; A61K 51/06

(57) ABSTRACT

A pharmaceutical formulation including a chelator-somatostatin receptor ligand and a transchelator is provided. The chelator-somatostatin receptor ligand is conjugated with a metal source or a radionuclide source, whereas the transchelator is capable of capturing free metal source or radionuclide source that is not conjugated to the chelator-somatostatin receptor ligand. By using such pharmaceutical formulation, the preparation of radiolabeled somatostatin analogues could be made more efficient, and is feasible for imaging of SSTR pathway-activated systems in cancers and neurological diseases.

### BACKGROUND OF THE INVENTION

### 1. Field of the Invention

The present invention generally relates to a pharmaceutical formulation, in particular, to a pharmaceutical formulation comprising a chelator-somatostatin receptor ligand mixed with a transchelator, and a method of preparing the same.

### 2. Description of Related Art

The somatostatin receptor (SSTR) pathway is the primary route of G-protein degradation in mammalian cells, which consists of a cascade of immune enzymatic reactions eventually leading to receptor tyrosine kinase related angiogenesis (VEGF, PDGFR) and cell proliferation. The over-expression of SSTR has been well documented in various tumors and neurological diseases. Most tumors carrying SSTR may express multiple SSTR subtypes, while the SSTR2 subtype is most predominantly expressed. The somatostatin analogue, octreotide, binds with high affinity to the SSTR2 and SSTR5 subtype while having a low affinity to the SSTR3 subtype. At present, ¹⁸F-fluorodopa, ⁶⁸Ga-DOTATATE, ¹¹¹In-Octreotide and ¹²³I-m-iodoguanidine along with biopsy specimen are the tools to identify neuroendocrine tumors. However, these ligands require either organic reaction or multi-step purification which are unsuitable for cGMP ready-to-use production.

In general, radiopharmaceutical chemistry requires intricate handling of radioactive materials, fast reaction times, ease of synthesis and reproducible results. In the preclinical setting, radiopharmaceuticals are typically synthesized manually. Such applications use in vitro and small animal models to validate the agent and require low levels of radioactivity. The use of manual synthesis for clinical imaging, however, is challenging for multiple reasons: 1) clinical agents must meet strict sterility and pyrogen requirements which are validated from batch to batch; 2) batch-to-batch reproducibility is required to demonstrate suitable radiochemical yield, radiochemical purity and other quality control analysis; 3) synthesis time must be fast when dealing with radionuclides with a short half-life; 4) clinical studies require multiple patient doses and would expose radiochemists to much higher levels of radioactivity; and 5) production cost and availability of the technology may limit the viability of the agent in routine clinical practice. The Food and Drug Administration (FDA) permits radiopharmaceuticals produced under well-controlled conditions in central commercial facilities to be distributed to local clinics where they are administered. In addition, radionuclide generator systems produced in well-controlled facilities have gained FDA acceptance and have a long history of successful clinical application. The clinical application of generator-based radiotracers is limited by the half-life of produced (daughter) radioisotopes and the choices of imaging agents. At present time, the preparation of radiolabeled somatostatin analogues requires column purification and the product is generally situated in organic solvents or ethanol/saline solution. There is therefore a need to overcome the current limitations in producing radiolabeled somatostatin analogues for clinical use.

### SUMMARY OF THE INVENTION

Accordingly, the present invention is directed to a pharmaceutical formulation having a chelator-somatostatin eceptor ligand, wherein no column purification step is required during its production.

The invention provides a pharmaceutical formulation including a chelator-somatostatin receptor ligand and a transchelator. The chelator-somatostatin receptor ligand is conjugated with a metal source or a radionuclide source, and the transchelator is capable of capturing free metal source or radionuclide source that is not conjugated to the chelator-somatostatin receptor ligand.

In an embodiment of the invention, the chelator-somatostatin receptor ligand is used as an active ingredient.

In an embodiment of the invention, the radionuclide source is selected from the group of metal ions including ^{99m}Tc, ^{117m}Sn, ¹⁷⁷Lu, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²²³Ra, ²¹²Bi, ²¹¹At, ⁴⁵Ti, ⁶⁰Cu, ⁶¹Cu, ⁶⁷Cu, ⁶⁴Cu and ⁶²Cu, and wherein the metal source is a non -radioactive metal such as ¹⁸⁷Re, ⁶⁹Ga, ¹⁵³Pt.

100081 In an embodiment of the invention, the chelator-somatostatin receptor ligand is octreotide ligands selected from DOTA-TOC, DOTATATE, DOTA-NOC or DTPAOC.

In an embodiment of the invention, the transchelator is citrate, mannitol, alpha-cyclodextrin, beta-cyclodextrin, gamma-cyclodextrin, hydroxypropyl cyclodextrin, glucose, glucosamine, gluconate, glucarate, glucoheptonate.

In an embodiment of the invention, the chelator-somatostatin receptor ligand is DOTATATE and the transchelator is beta-cyclodextrin, wherein based on 100 μg of DOTATATE, a usage amount of the beta-cyclodextrin is in a range from 1 mg to 100 mg, and a pH of the pharmaceutical formulation is between 4 to 5.

The invention further provides a method of preparing a pharmaceutical formulation including the following steps. A chelator-somatostatin receptor ligand was reacted with a metal source or a radionuclide source so that the metal source or the radionuclide source is conjugated to the chelator-somatostatin receptor ligand, wherein no column purification step is performed after reacting the chelator-somatostatin receptor ligand with the metal source or the radionuclide source. The chelator-somatostatin receptor ligand was mixed with a transchelator.

In an embodiment of the invention, the pharmaceutical formulation is prepared into one of the following forms for administration: tablets, capsules, powders, dispersible granules, cachets and suppositories, sustained release and delayed release formulations, liquid dosage forms, solutions, suspensions and emulsions, injectable formulations, solutions or sprays for intranasal, buccal or sublingual administration, aerosol preparations suitable for inhalation, transdermal formulations, creams, lotions, aerosols and/or emulsions and transdermal patches.

In an embodiment of the invention, the pharmaceutical formulation is administered intravenously.

In an embodiment of the invention, the radionuclide source is selected from the group of metal ions including ^{99m}Tc, ^{117m}Sn, ¹⁷⁷Lu, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²²³Ra, ²¹²Bi, ²¹¹At, ⁴⁵Ti, ⁶⁰Cu, ⁶¹Cu, ⁶⁷Cu, ⁶⁴Cu and ⁶²Cu, and wherein the metal source is a non-radioactive metal such as ¹⁸⁷Re, ⁶⁹Ga, ¹⁵³Pt.

In an embodiment of the invention, the chelator-somatostatin receptor ligand is octreotide ligands selected from DOTA-TOC, DOTATATE, DOTA-NOC or DTPAOC.

In an embodiment of the invention, the transchelator is citrate, mannitol, alpha-cyclodextrin, beta-cyclodextrin gamma-cyclodextrin, hydroxypropyl cyclodextrin, glucose, glucosamine, gluconate, glucarate, glucoheptonate.

In an embodiment of the invention, the chelator-somatostatin receptor ligand is DOTATATE and the transchelator is beta-cyclodextrin, wherein based on 100 μg of DOTATATE, a usage amount of the beta-cyclodextrin is in a range from 1 mg to 100 mg, and a pH of the pharmaceutical formulation is between 4 to 5.

The invention further provides a method of imaging neuroendocrine tumor in a patient using nuclear imaging, wherein the method comprises administering to the patient an effective amount of the pharmaceutical formulation noted above, wherein the chelator-somatostatin receptor ligand conjugated with the metal source or the radionuclide source is ⁶⁸Ga-DOTATATE or ^{99m}Tc-DOTATATE; and the transchelator is beta-cyclodextrin.

In an embodiment of the invention, the neuroendocrine tumor is selected from the group consisting of brain tumor, breast tumor, prostate tumor, colon tumor, lung tumor, liver tumor, pancreas tumor, gastric tumor, lymphoma, uterine tumor, cervical tumor, thyroid tumor and melanoma.

In an embodiment of the invention, the nuclear imaging used is positron emission tomography (PET) or single photon emission computed tomography (SPECT).

The invention further provides a method of imaging somatostatin receptor system in a patient with neurological diseases and psychiatric disorder using nuclear imaging, wherein the method comprises administering to the patient an effective amount of the pharmaceutical formulation noted above.

In an embodiment of the invention, the neurological diseases and the psychiatric disorder are selected from the group consisting of Alzheimer, Huntington's disease, Parkinson, Epilepsy, Amyotrophic lateral sclerosis (ALS), Posttraumatic stress disorder (PTSD), Attention deficit hyperactivity disorder (ADHD), dementia, mood disorders and psychic symptoms.

In an embodiment of the invention, the nuclear imaging used is positron emission tomography (PET) or single photon emission computed tomography (SPECT).

Based on the above, the pharmaceutical formulation of the present invention includes a chelator-somatostatin receptor ligand and a transchelator, wherein the transchelator is capable of capturing free metal source or radionuclide source that is not conjugated to the chelator-somatostatin receptor ligand. Since the transchelator is capable of capturing free/unbound metal source or radionuclide source, thus column purification steps can be avoided, and the preparation of radiolabeled somatostatin analogues could be made more efficient, and be feasible for imaging of SSTR pathway-activated systems in cancers and neurological diseases.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a HPLC diagram of cold Ga-DOTATATE prepared in Example 1 of the invention.

FIG. 2A is an ITLC diagram of ⁶⁸Ga-DOTATATE prepared in Example 1 of the invention.

FIG. 2B is a HPLC diagram of ⁶⁸Ga-DOTATATE prepared in Example 1 of the invention.

FIG. 2C is an ITLC diagram of ⁶⁸Ga-DOTA prepared in Example 1 of the invention.

FIG. 2D is a HPLC diagram of ⁶⁸Ga-DOTA prepared in Example 1 of the invention.

FIG. 3A is a HPLC diagram of ⁶⁸Ga-DOTATATE obtained using a NaI detector according to Example 1 of the invention.

FIG. 3B is a HPLC diagram of ⁶⁸Ga-DOTATATE obtained using an UV detector according to Example 1 of the invention.

FIG. 4A is a HPLC diagram of ⁶⁸Ga-CD and ⁶⁸Ga-DOTATATE obtained using a NaI detector according to Example 1 of the invention.

FIG. 4B is a HPLC diagram of ⁶⁸Ga-CD and ⁶⁸Ga-DOTATATE obtained using an UV detector according to Example 1 of the invention.

FIG. 5A is an ITLC diagram of ⁶⁸Ga-CD using an acetone system according to Example 1 of the invention.

FIG. 5B is an ITLC diagram of ⁶⁸Ga-CD using a saline system according to Example 1 of the invention.

FIG. 6 is a PET/CT image of a human colorectal-tumor bearing mouse administered with ¹⁸F-FDG according to Example 2 of the invention.

FIG. 7 is a PET/CT image of a human pancreatic-tumor bearing mouse administered with ¹⁸F-FDG according to Example 2 of the invention.

FIG. 8 is a PET/CT image of a human colorectal-tumor bearing mouse administered with ⁶⁸Ga-DOTATATE/CD according to Example 2 of the invention.

FIG. 9 is a PET/CT image of a human pancreatic-tumor bearing mouse administered with ⁶⁸Ga-DOTATATE/CD according to Example 2 of the invention.

FIG. 10 is a PET/CT image of a human colorectal-tumor bearing mouse administered with ⁶⁸Ga-DOTATATE according to Example 2 of the invention.

FIG. 11 is a PET/CT image of a human pancreatic-tumor bearing mouse administered with ⁶⁸Ga-CD according to Example 2 of the invention.

FIG. 12 is a PET/CT image of a human pancreatic-tumor bearing mouse administered with ⁶⁸Ga-DOTA according to Example 2 of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

The present invention provides a novel formulation using a transchelator for preparing metallic chelator-octreotide conjugates and its application in oncology and neurology imaging and therapy. For example, a pharmaceutical formulation of the invention includes a chelator-somatostatin receptor ligand and a transchelator. The chelator-somatostatin receptor ligand is conjugated with a metal source or a radionuclide source. In an embodiment of the invention, the chelator-somatostatin receptor ligand is octreotide ligands selected from DOTA-TOC (formula IA), DOTATATE (formula IB), DOTA-NOC (formula IC) or diethylene triamine penta-acetic acid octreotide (DTPAOC).

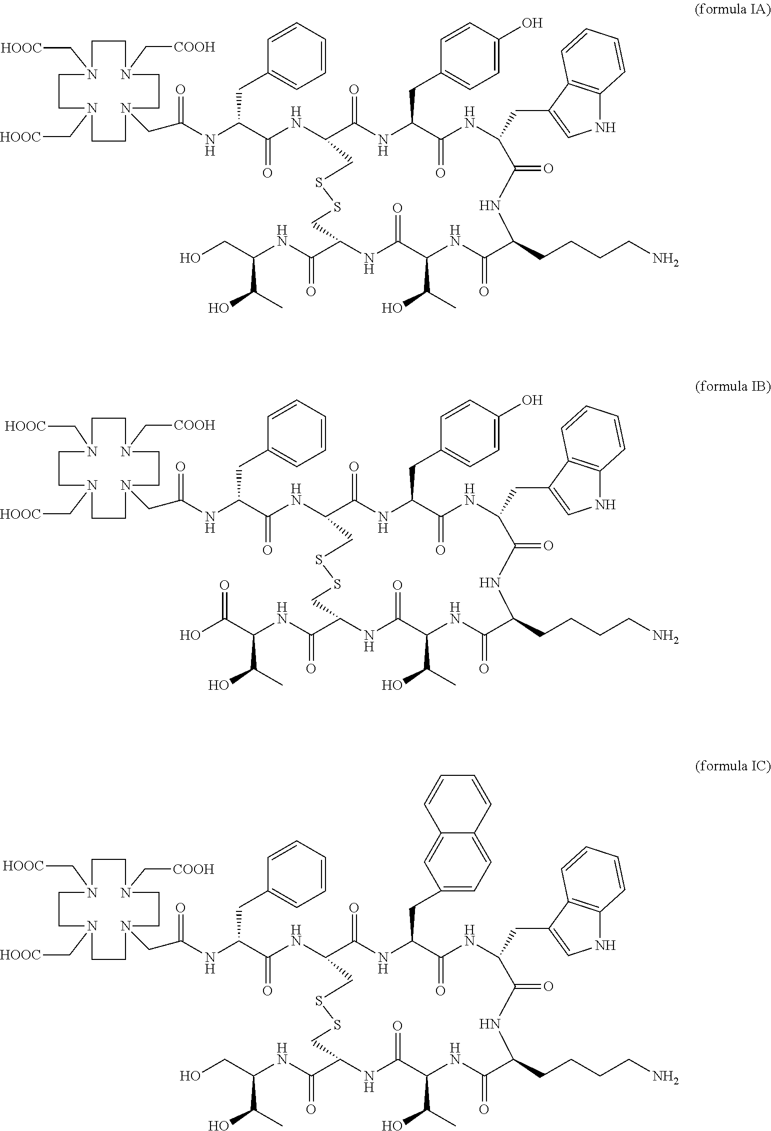

The chelator-somatostatin receptor ligand used in the present invention is for example, an inverse agonist of the SSTR and the subtype of SSTR 1-5. Preferably, the chelator-somatostatin receptor ligand is DOTATATE (formula IB). The chelator-somatostatin receptor ligand could be reacted with radionuclide/metal source to form metallic complexes. In an embodiment of the invention, the radionuclide source is selected from the group of metal ions including ^{99m}Tc, ^{117m}Sn, ¹⁷⁷Lu, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²²³Ra, ²¹²Bi, ²¹¹At, ⁴⁵Ti, ⁶⁰Cu, ⁶¹Cu, ⁶⁷Cu, ⁶⁴Cu and ⁶²Cu, and wherein the metal source is a non-radioactive metal such as ¹⁸⁷Re, ⁶⁹Ga, ¹⁵³Pt. In a particular example, the radionuclide source is gallium, and DOTATATE is reacted with gallium chloride to foam a complex shown in formula ID.

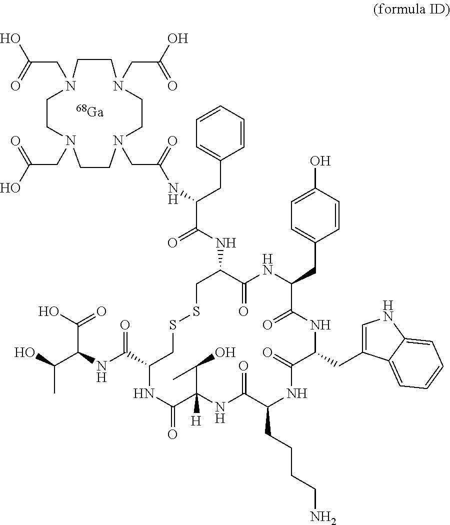

In addition, the transchelator added in the pharmaceutical formulation of the invention is capable of capturing free metal source or radionuclide source that is not conjugated to the chelator-somatostatin receptor ligand. In certain embodiments, the transchelator is for example, citrate, mannitol, alpha-cyclodextrin (formula IIA), beta-cyclodextrin (formula IIB), gamma-cyclodextrin (formula IIC), hydroxypropyl cyclodextrin, glucose, glucosamine, gluconate, glucarate, glucoheptonate. Preferably, the transchelator is beta-cyclodextrin (formula IIB).

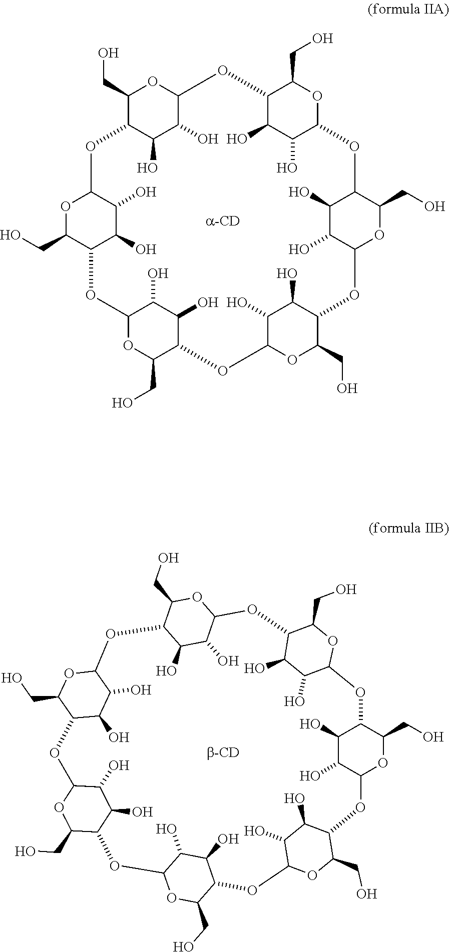

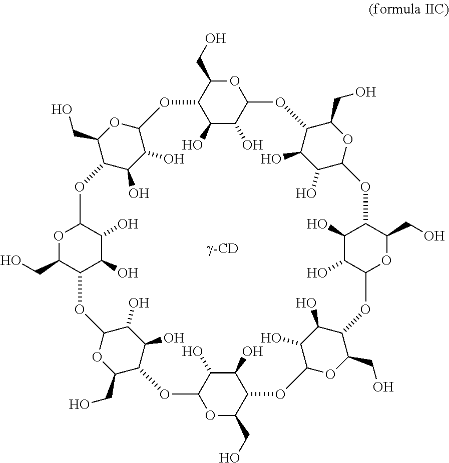

In a particular example, when the chelator-somatostatin receptor ligand is DOTATATE and the transchelator is beta-cyclodextrin, then based on 100 μg of DOTATATE, a usage amount of the beta-cyclodextrin is in a range from 1 mg to 100 mg, and a pH of the pharmaceutical formulation is between 4 to 5. By using the DOTATATE and the beta-cyclodextrin transchelator in such ratio, the beta-cyclodextrin would be successful in capturing free metal source or radionuclide source not conjugated to the chelator-somatostatin receptor ligand. The transchelator may act as a subtype of the SSTR 1-5 ligand, hence, capturing free metal source or radionuclide source. In certain embodiments, the metal source or radionuclide source captured by the transchelator (e.g. beta-cyclodextrin) may also be released back to the chelator-somatostatin receptor ligand for conjugation.

The method of preparing the pharmaceutical formulation of the invention includes reacting the chelator-somatostatin receptor ligand with a metal source or a radionuclide source so that the metal source or the radionuclide source is conjugated to the chelator-somatostatin receptor ligand. In particular, no column purification step is performed after reacting the chelator-somatostatin receptor ligand with the metal source or the radionuclide source. Subsequently, the labelled chelator-somatostatin receptor ligand is mixed with the transchelator in an aqueous solvent, such as saline or sterilized water.

In general, the method of preparing the pharmaceutical formulation of the invention requires no column purification step. However, compounds of the present invention may be purified via any method known to those skilled in the art when required. It should be noted that persons skilled in the art should be familiar with the methods of purification, and when these purification methods may be employed. For example, in a multi-step synthesis that is aimed at arriving at a particular compound, a purification step may be performed after every synthetic step, after every few steps, at various points during the synthesis, and/or at the very end of the synthesis. In some methods, one or more purification steps comprises technique selected from the group consisting of silica gel column chromatography, C-18 reverse phase column chromatography, gel permeation column chromatography, HPLC (high-performance liquid chromatography) and LC (liquid chromatography). In certain embodiments, purification methods specifically exclude size exclusion chromatography and/or dialysis. In a particular aspect, the method may comprise purifying a chelator-octreotide after the metallic incorporation.

In certain embodiments, the pharmaceutical formulation is prepared into one of the following forms for administration: tablets, capsules, powders, dispersible granules, cachets and suppositories, sustained release and delayed release formulations, liquid dosage forms, solutions, suspensions and emulsions, injectable formulations, solutions or sprays for intranasal, buccal or sublingual administration, aerosol preparations suitable for inhalation, transdermal formulations, creams, lotions, aerosols and/or emulsions and transdermal patches. Preferably, the pharmaceutical formulation is administered intravenously.

Further embodiments of the invention concern methods of imaging a site, diagnosing a disease, or treating a disease within a subject. The method may comprise obtaining a metal ion labelled conjugate prepared as described herein and administering to the subject with a metal ion conjugate, wherein the site is imaged, the staging of the disease is diagnosed, or the disease is treated. The signal may be detected using a technique selected from the group consisting of positron emission tomography (PET), PET/computed tomography (CT), single-photon emission computed tomography (SPECT), SPECT/CT, PET/magnetic resonance imaging (MRI) and SPECT/MRI. The site to be imaged may be tumors or brain. The method may be further defined as a method of imaging, diagnosing, or treating a subject with cancers and neurodegenerative diseases. In particular aspects, the cancer is carcinoid, neuroendocrine cancer, breast cancer, lung cancer, prostate cancer, ovarian cancer, brain cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, thyroid cancer, lymphoma, or leukemia.

More particularly, the invention is concerned with a method of imaging neuroendocrine tumor in a patient using nuclear imaging such as PET or SPECT. The method comprises administering to the patient an effective amount of the pharmaceutical formulation as described above. In particular, the chelator-somatostatin receptor ligand conjugated with the metal source or the radionuclide source is ⁶⁸Ga-DOTATATE or ^{99m}Tc-DOTATATE; and the transchelator is beta-cyclodextrin. The neuroendocrine tumor is selected from the group consisting of brain tumor, breast tumor, prostate tumor, colon tumor, lung tumor, liver tumor, pancreas tumor, gastric tumor, lymphoma, uterine tumor, cervical tumor, thyroid tumor and melanoma.

Additionally, the invention is also concerned with a method of imaging somatostatin receptor system in a patient with neurological diseases and psychiatric disorder using nuclear imaging such as PET or SPECT, wherein the method comprises administering to the patient an effective amount of the pharmaceutical formulation described above. More particularly, the neurological diseases and the psychiatric disorder are selected from the group consisting of Alzheimer, Huntington's disease, Parkinson, Epilepsy, Amyotrophic lateral sclerosis (ALS), Posttraumatic stress disorder (PTSD), Attention deficit hyperactivity disorder (ADHD), dementia, mood disorders and psychic symptoms.

To prove that the pharmaceutical formulation of the invention is more suitable for imaging of the SSTR pathway-activated systems in cancers and neurological diseases and can be prepared with higher efficiency, the pharmaceutical formulation is synthesized and tested by using the method described in the following examples.

### EXAMPLE 1

### Synthesis of ⁶⁸Ga-DOTATATE

Determination of ⁶⁸ Ga-Elusion Profile from a ⁶⁸Ge/⁶⁸Ga Generator

In this example, ⁶⁸GaCl₃ was obtained from a ⁶⁸Ge/⁶⁸Ga generator eluted with HCl (ranging from 0.01N-1N). For instance, ⁶⁸GaCl₃ was eluted from a ⁶⁸Ge/⁶⁸Ga generator with 0.3N and 0.6N HCl (10 mL). On the following day, the elusion volume (0.3N or 0.6N HCl, 6 mL) was distributed in a 12-tube (0.5 mL/tube). Each tube was counted for its radioactivity and the results are shown in Table 1 below.

TABLE 1
⁶⁸Ga Elusion Activity Profile

| | Elute 1 (0.6N HCl_{(aq)}) | Elute 2 (0.3N HCl_{(aq)}) |
|---|---|---|
| Fraction 1 | 0.41 uCi | 0.79 mCi |
| Fraction 2 | 0.35 uCi | 0.95 mCi |
| Fraction 3 | 0.903 mCi | 7.22 mCi |
| Fraction 4 | 7.87 mCi | 3.10 mCi |
| Fraction 5 | 7.55 mCi | 2.45 mCi |
| Fraction 6 | 6.25 mCi | 1.51 mCi |
| Fraction 7 | 0.851 mCi | 0.73 mCi |
| Fraction 8 | 0.381 mCi | 0.637 mCi |
| Fraction 9 | 0.470 mCi | 0.431 mCi |
| Fraction 10 | 0.770 mCi | 0.441 mCi |
| Fraction 11 | 0.804 mCi | 0.324 mCi |
| Fraction 12 | 0.482 mCi | 0.444 mCi |
| Total | 26.331 mCi | 20.427 mCi |

As shown in Table 1, the highest activities are between fractions 4 to 6, wherein these fractions are selected and combined. In the consecutive cycle, the generator is eluted again with 6 mL HCl and collected at these specific fractions.

### Synthesis of Cold Ga-DOTATATE

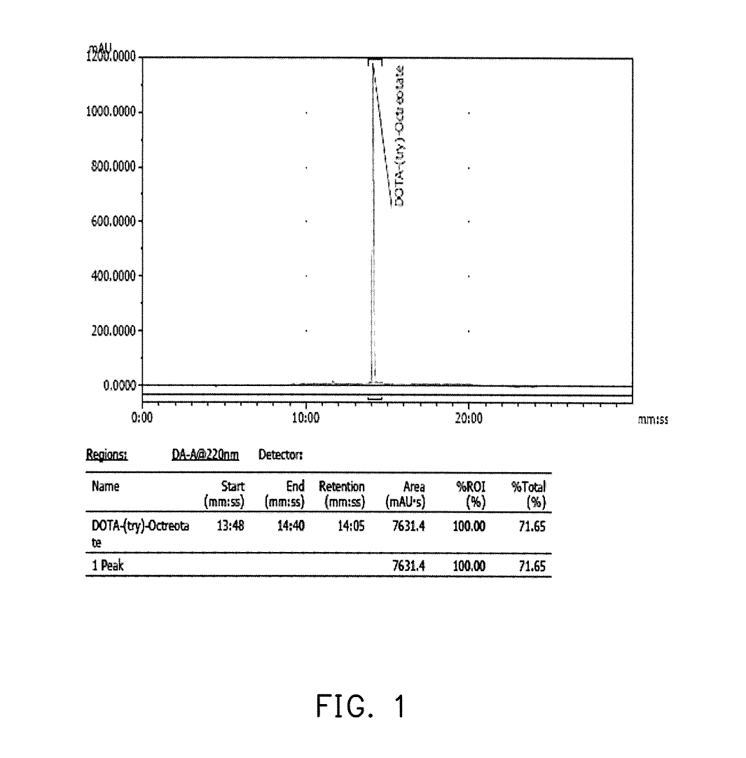

Specifically, ⁶⁹GaCl₃ (61 mL, 1 mg/mL in 0.05M HCl) was added to a solution of DOTATATE (100 mg, 0.07 mmol) in 0.25 ml ammonium acetate (NH₄OAc; 0.4M). The solution was heated for 25 min at 95° C. The product was purified by a C-18 Sepak column eluting with saline (5 mL) to remove free gallium (Ga). The product was then collected with ethanol (3 mL) to yield Ga-DOTATATE. After solvent evaporation, Ga-DOTATATE was obtained as a white solid (100 mg). High performance liquid chromatograph (HPLC) was used to confirm the structure of ⁶⁹Ga-DOTATATE, and the results are presented in FIG. 1. As shown in FIG. 1, retention time for ⁶⁹Ga-DOTATATE (C-18 column, 20 uL injection, 220-280 nm) at 0.6 mL/min is 14 min. The HPLC showed only a sharp single peak for ⁶⁹Ga-DOTATATE, suggesting high purity.

Radiosynthesis of ⁶⁸Ga-DOTATATE with Column Purification

### Method 1 (Using Sodium Acetate to Adjust pH):

In the following method, ⁶⁸GaCl₃ (1.5 ml in 0.6N HCl, 20.1 mCi) was eluted from a commercial generator (2^{nd} fraction, 1.5 mL/fraction) based upon previous elusion profile. An aliquot of ⁶⁸GaCl₃ (0.5 ml in 0.6N HCl, 6.70 mCi) was added to the solution of DOTATATE (0.1 mg) in 0.8 ml sodium acetate (NaOAc; 2.5M), and pH value was 4-5. The solution was heated at 70° C. for 10 min. After cooling, the reaction mixture was loaded onto a C-18 Sepak column which was pre-activated by ethanol (5 mL) and saline (5 mL). The column was eluted with ethanol (30%, 1 mL), followed by saline (2 mL) to yield the desired product (2.66 mCi, 40%, no decay correction, EOS 30 min). High-performance liquid chromatography (HPLC), equipped with a NaI detector and UV detector (280 nm), was performed on a C-18 reverse phase column (C18-extend, Agilent, Santa Clara, Calif.) eluted with acetonitrile/water (gradient) containing 0.1% trifluoroacetic acid at a flow rate of 0.5 ml/min.

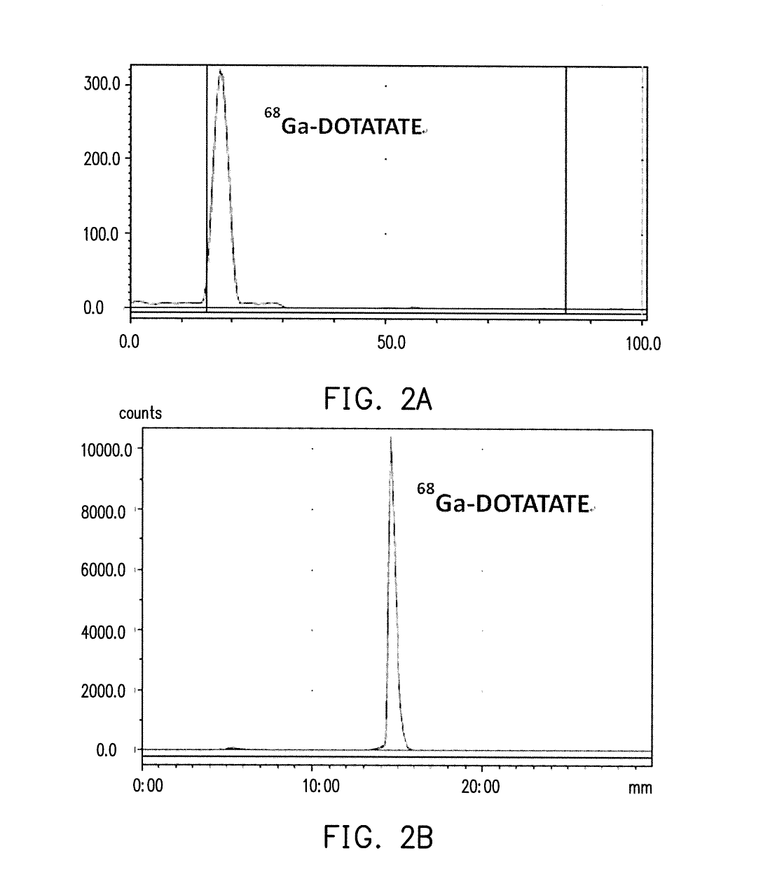

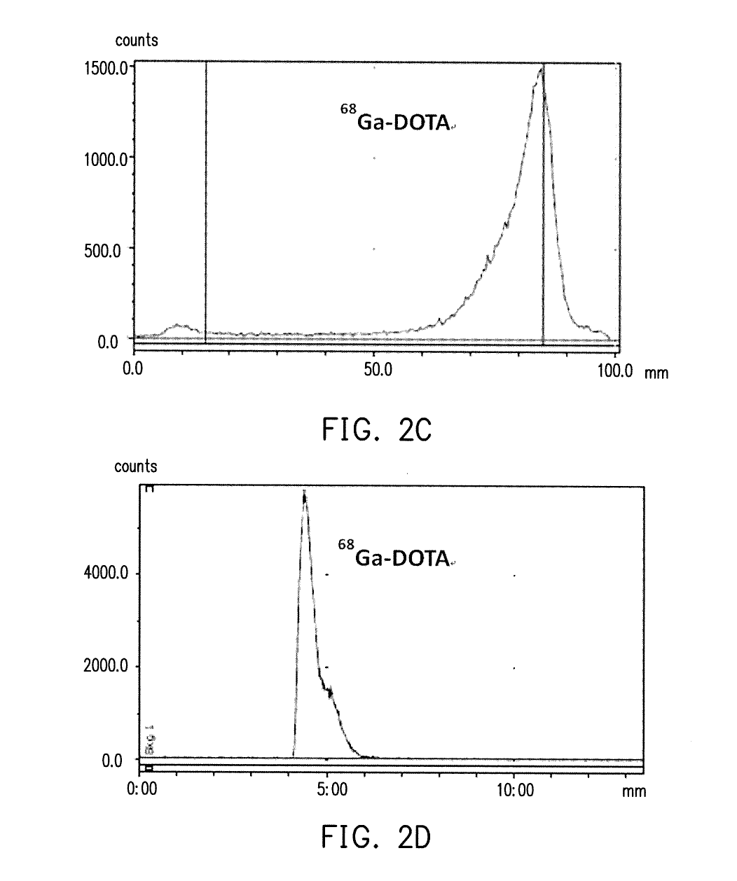

Radiochemical purity was determined by ITLC (Waterman No.1, Aldrich-Sigma, St. Louis, Mo.) eluted with saline, and the results are presented in FIG. 2A. Furthermore, HPLC of cold ⁶⁹Ga-DOTATATE was used to confirm the structure of ⁶⁸Ga-DOTATATE, and the analysis results are presented in FIG. 2B. As shown in FIGS. 2A and 2B, by using the sodium acetate method, the radiochemical purity was 100% with a Rf value of 0.2, and the retention time of ⁶⁸Ga-DOTATATE showed 14 min which matched the peak of ⁶⁹Ga-DOTATATE. Furthermore, the ITLC and HPLC analysis of ⁶⁸Ga-DOTA prepared under the same conditions are evaluated and the results are shown in FIGS. 2C and 2D. As shown in FIGS. 2C and 2D, the retention time and Rf of ⁶⁸Ga-DOTA were 5 min and 0.8, respectively.

### Method 2 (Using Sodium Bicarbonate to Adjust pH):

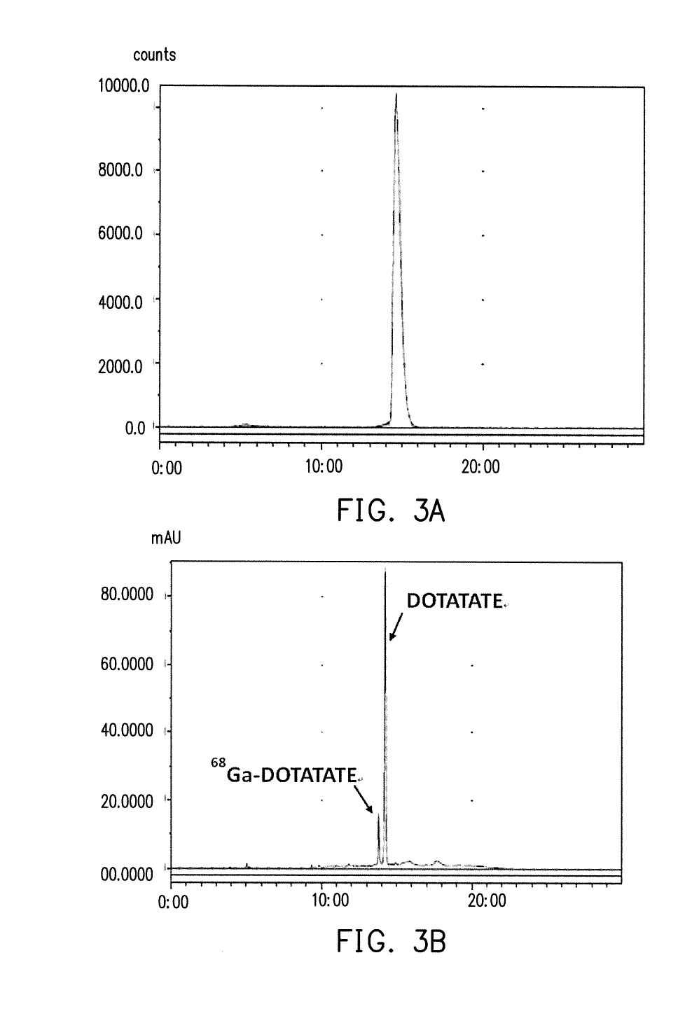

In the following method, ⁶⁸GaCl₃ (1.5 ml in 0.6N HCl, 16.58 mCi) was eluted from a commercial generator (2^{nd} fraction, 1.5 mL/fraction) based upon previous elusion profile. An aliquot of ⁶⁸GaCl₃ (0.5 ml in 0.6N HCl, 6.46 mCi) was added to the solution of DOTATATE (0.1 mg) in 0.2 ml ammonium acetate (0.4M), and pH value was adjusted to 4-5 with sodium bicarbonate (NaHCO₃; 0.32 ml, 1N). The solution was heated at 70° C. for 10 min. After cooling, the reaction mixture was loaded on to a C-18 Sepak column which was pre-activated by ethanol (5 mL) and saline (5 mL). The column was eluted with ethanol (30%, 1 mL), followed by saline (2 mL) to yield the desired product (1.48 mCi, 23%, no decay correction, EOS 30 min). High-performance liquid chromatography (HPLC), equipped with a NaI detector and UV detector (280 nm), was performed on a C-18 reverse phase column (C18-extend, Agilent, Santa Clara, Calif.) eluted with acetonitrile/water (gradient) containing 0.1% trifluoroacetic acid at a flow rate of 0.5 ml/min. HPLC of cold ⁶⁹Ga-DOTATATE was used to confirm the structure of ⁶⁸Ga-DOTATATE. The HPLC analysis results are presented in FIG. 3A and FIG. 3B. As shown in FIGS. 3A and 3B, by using the sodium bicarbonate method, the radiochemical purity was high and the retention time of ⁶⁸Ga-DOTATATE showed 14 min which matched the peak of ⁶⁹Ga-DOTATATE.

### Radiosynthesis of ⁶⁸Ga-DOTATATE Using a Transchelator (Without Column Purification)

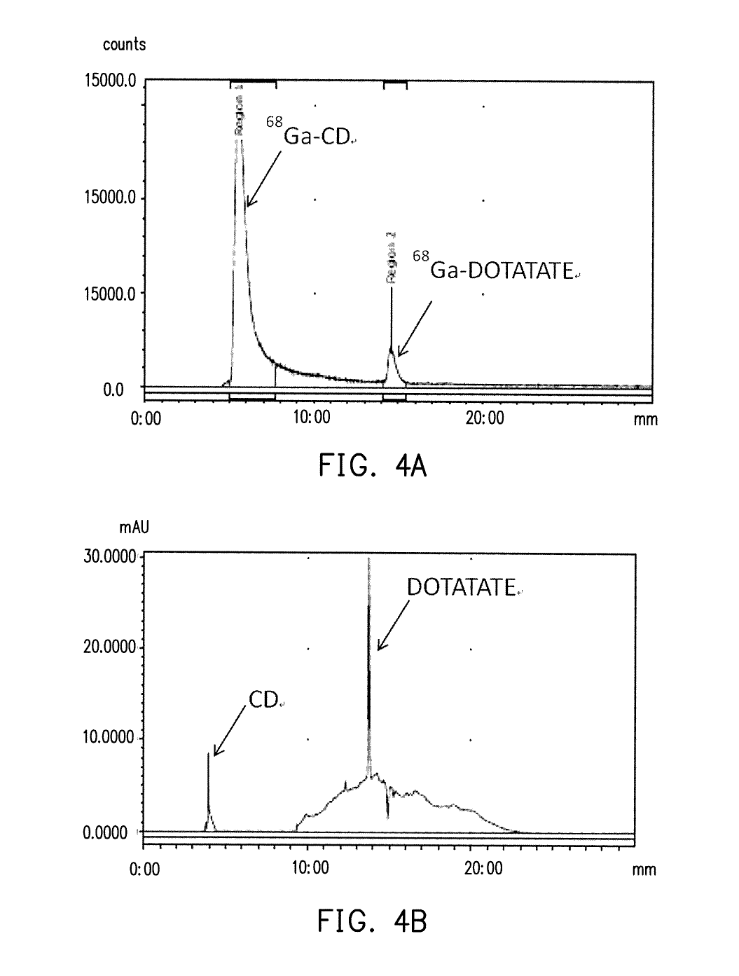

Specifically, ⁶⁸GaCl₃ was obtained from a ⁶⁸Ge/⁶⁸Ga generator eluted with HCl (0.6N, 6 mL). The first 1.5 mL (0.2 mCi) was discarded. The second 1.5 mL ⁶⁸GaCl₃ (13.54 mCi) was added to the 800 μL 2.5M NaOAc or 1 mL 1N NaHCO₃ containing DOTATATE (0.1 mg) and beta-cyclodextrin (CD, 1.3 mg; transchelator). The pH value was approximately 4-5. The reaction mixture was heated to 70° C. for 10 min. after heating, the pH was re-adjusted to 6-7 by 1 mL 1N NaHCO₃. The product was filtered through a 0.22 μM filter, yielded 9.72 mCi (72%, no decay correction, EOS 15 min). HPLC equipped with a NaI detector and UV detector (280 nm) was used for analysis, and the analysis results are presented in FIG. 4A and FIG. 4B. As shown in FIG. 4A, by using the NaI detector, two peaks can be observed for ⁶⁸Ga-CD and ⁶⁸Ga-DOTATATE, suggesting that the beta-cyclodextrin (CD) is capable of capturing the free ⁶⁸Ga that is not conjugated to DOTATATE. Furthermore, by using the UV detector, HPLC analysis showed that the retention time of ⁶⁸Ga-CD and ⁶⁸Ga-DOTATATE were 5 min and 14 min, respectively.

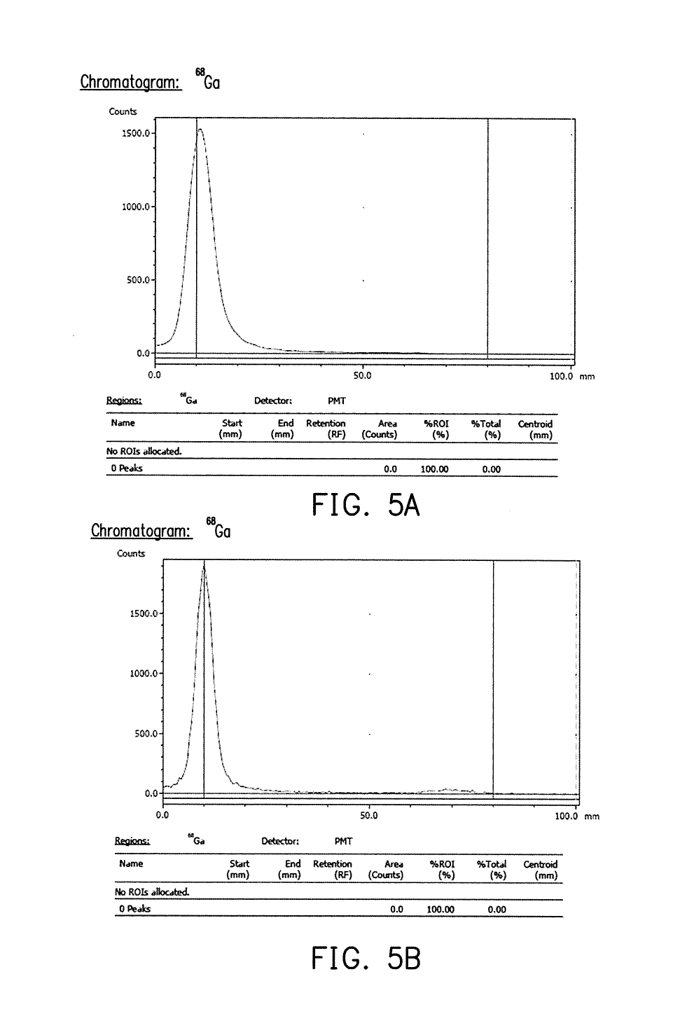

In addition, two mobile phases were used for the analysis of ⁶⁸Ga-Beta-cyclodextrin (⁶⁸Ga-CD) as presented in FIG. 5A and FIG. 5B. FIG. 5A is the ITLC diagram of ⁶⁸Ga-CD using an acetone system, while FIG. 5B is the ITLC diagram of ⁶⁸Ga-CD using a saline system. ITLC analysis showed that the Rf value of ⁶⁸Ga-CD in both systems was 0.2, which is with the same Rf value as ⁶⁸Ga-DOTATATE.

Moreover, various amounts of beta-cyclodextrin (1.3 mg-10 mg) was mixed with ⁶⁸Ga-DOTATATE and was tested for its activity and yield, and the results are shown in Table 2.

TABLE 2
⁶⁸Ga-DOTATATE (0.1 mg) with CD (1.3 mg-10 mg)

| | Date | | |
|---|---|---|---|
| | 2016 Jul. 12 | 2016 Jul. 13 | 2016 Jul. 15 |
| DOTATAE (μg) | 100 | 100 | 100 |
| Cyclodextrin (μg) | 10000 | 1600 | 1300 |
| 2.5N NaOAc (mL) | 0.8 | 0.8 | 0.8 |
| 1N NaHCO₃ (mL) | 1 | 1 | 1 |
| Ga extraction | 13.59 | 15.91 | 13.54 |
| activity (mCi) | | | |
| Ga extraction time | 10:40 | 14:43 | 15:55 |
| Product generation | 9.03 | 11.33 | 9.72 |
| activity | | | |
| Product generation | 10:57 | 14:57 | 16:06 |
| time | | | |
| Yield | 79.01796 | 82.13656 | 80.30519 |
| (attenuation | | | |
| correction)* | | | |

*The yield is calculated based on the final solution activity

From the results shown above, the transchelator (beta-cyclodextrin) may be considered as an “excipient” (inactive pharmaceutical ingredient), while the DOTATATE may be treated as the active ingredient. In summary, ITLC showed that ⁶⁸Ga-CD is located at nearly the same Rf and retention time as free ⁶⁸Ga using acetone and saline systems, unlike ⁶⁸Ga-DOTA. On the other hand, HPLC data showed distinguishable peaks between ⁶⁸Ga-DOTATATE (14 min) and ⁶⁸Ga-CD (5 min). The optimal ratio that is required for the transchelator (CD; approximately 1 mg-10 mg) and the chelator-somatostatin receptor ligand (DOTATATE; 100 μg) to show sufficient activity is also presented in Table 2. By using such chelator-somatostatin receptor ligand/transchelator ratio, all of the free/unbound ⁶⁸Ga can be captured. As such, by using cyclodextrin as a transchelator in the preparation of ⁶⁸Ga-DOTATATE avoids column purification, and produced sufficient activity for biologic studies.

### EXAMPLE 2

### Positron Emission Tomographic (PET) Imaging Studies

To prove that the formulation of mixing ⁶⁸Ga-DOTATATE with a transchelator beta-cyclodextrin (CD) has equal or better imaging quality than ⁶⁸Ga-DOTATATE alone, two known neuroendocrine tumor-bearing animal models (colorectal and pancreatic) were selected for imaging. Specifically, ⁶⁸Ga-N4-tyrosine will be incubated with plasma up to 3 hours. The imaging data were compared to ¹⁸F-FDG (gold standard) and ⁶⁸Ga-DOTA (negative control).

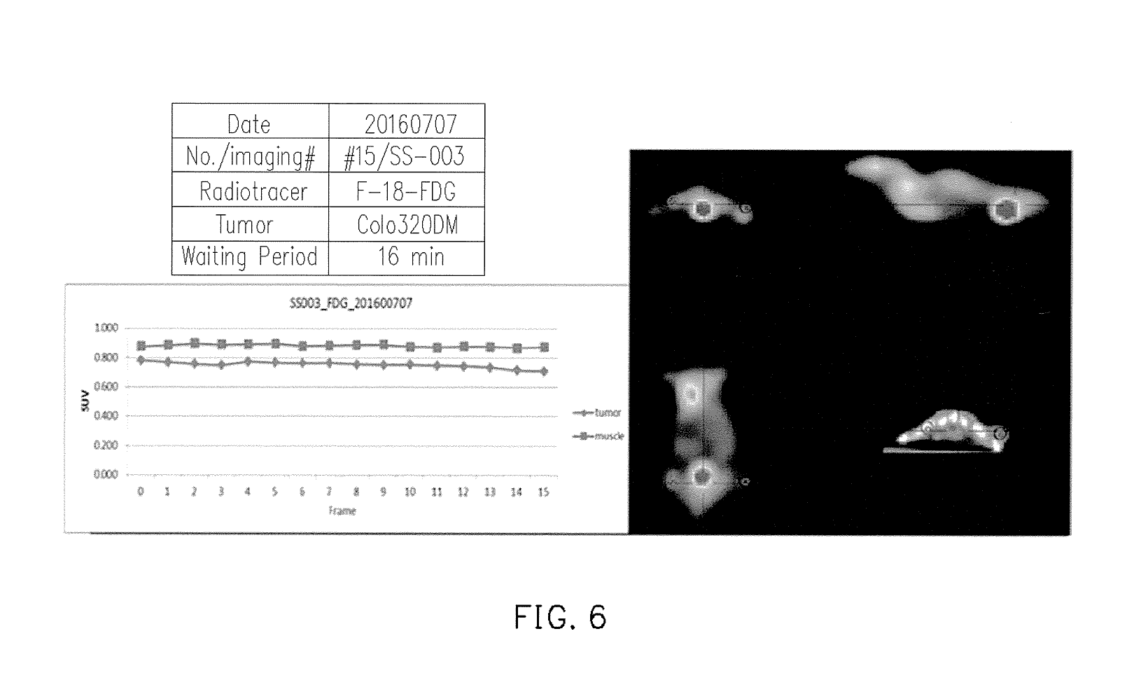

Briefly, athymic nude mice (15±2 g) bearing human tumors (at hind legs) derived from the colorectal and pancreatic cell line were used for imaging studies. Studies were performed 21 to 28 days after inoculation when tumors were approximately 0.5 cm in diameter. Scintigraphic images were obtained either from a micro-PET (Inveon) embedded in the gantries coordinate PET/CT data acquisition. Each animal was administered with ⁶⁸Ga-DOTATATE/CD, ⁶⁸Ga-DOTATATE, ⁶⁸Ga-DOTA or ¹⁸F-FDG (500 μCi/mouse, iv), and the dynamic images was from 0 to 30 minutes. The static images were obtained at 0.5, 1 and 2 hrs. Computer outlined regions of interest (ROI) (counts per pixel) for tumor and muscle at the corresponding time interval were used to generate a dynamic plot for ⁶⁸Ga-tracers and ¹⁸F-FDG. The analysis results are presented in FIGS. 6-12.

FIG. 6 is a PET/CT image of a human colorectal-tumor bearing mouse administered with ¹⁸F-FDG according to Example 2 of the invention. The average tumor/muscle (T/M) count density ratios for ¹⁸F-FDG in colorectal tumor models were calculated and presented in Table 3. Results from FIG. 6 and Table 3 indicated that the average tumor/muscle (T/M) count density ratios for ¹⁸F-FDG in colorectal tumor-bearing mice were approximately in the range of 0.8 to 0.9, which suggested a slightly higher muscle uptake as compared to tumor uptake. The results for ¹⁸F-FDG was used as a standard for comparison to other compounds/compositions in colorectal tumor models.

TABLE 3
Tumor/muscle (T/M) count density ratios for ¹⁸F-FDG in colorectal tumor models

| Frame | 0 | 1 | 2 | 3 | 4 | 5 | 6 | 7 | 8 |
|---|---|---|---|---|---|---|---|---|---|
| Duration (sec) | 15 | 15 | 15 | 15 | 30 | 30 | 60 | 60 | 60 |
| Midpoint (sec) | 15 | 30 | 45 | 60 | 90 | 120 | 180 | 240 | 300 |
| Tumor | 0.782 | 0.769 | 0.760 | 0.750 | 0.774 | 0.766 | 0.762 | 0.764 | 0.758 |
| Muscle | 0.879 | 0.887 | 0.900 | 0.892 | 0.892 | 0.897 | 0.881 | 0.885 | 0.890 |
| Tumor/Muscle ratio | 0.890 | 0.867 | 0.844 | 0.841 | 0.868 | 0.854 | 0.866 | 0.864 | 0.852 |

| Frame | 9 | 10 | 11 | 12 | 13 | 14 | 15 |
|---|---|---|---|---|---|---|---|
| Duration (sec) | 60 | 180 | 180 | 180 | 300 | 300 | 300 |
| Midpoint (sec) | 360 | 540 | 720 | 900 | 1200 | 1500 | 1800 |
| Tumor | 0.753 | 0.756 | 0.749 | 0.745 | 0.737 | 0.719 | 0.712 |
| Muscle | 0.891 | 0.877 | 0.872 | 0.879 | 0.877 | 0.869 | 0.874 |
| Tumor/Muscle ratio | 0.846 | 0.862 | 0.859 | 0.847 | 0.840 | 0.827 | 0.815 |

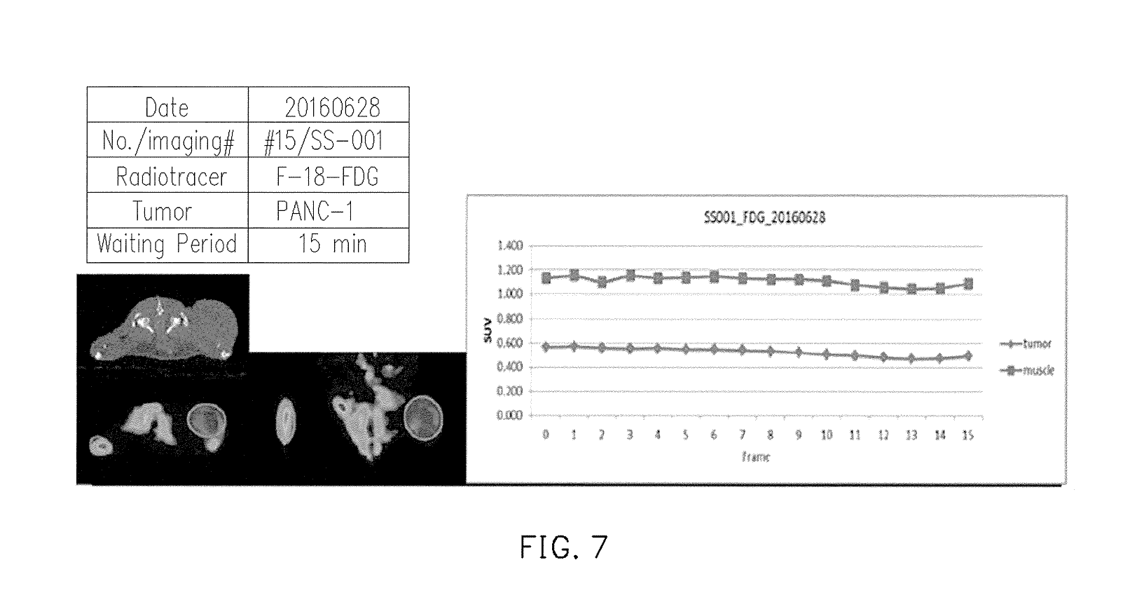

FIG. 7 is a PET/CT image of a human pancreatic-tumor bearing mouse administered with ¹⁸F-FDG according to Example 2 of the invention. The average tumor/muscle (T/M) count density ratios for ¹⁸F-FDG in pancreatic tumor models were calculated and presented in Table 4. Results from FIG. 7 and Table 4 indicated that the average tumor/muscle (T/M) count density ratios for ¹⁸F-FDG in pancreatic tumor-bearing mice were approximately in the range of 0.45 to 0.51, which suggested a much higher muscle uptake as compared to tumor uptake. The results for ¹⁸F-FDG was used as a standard for comparison to other compounds/compositions in pancreatic tumor models.

TABLE 4
Tumor/muscle (T/M) count density ratios for ¹⁸F-FDG in pancreatic tumor models

| Frame | 0 | 1 | 2 | 3 | 4 | 5 | 6 | 7 | 8 |
|---|---|---|---|---|---|---|---|---|---|
| Duration (sec) | 15 | 15 | 15 | 15 | 30 | 30 | 60 | 60 | 60 |
| Midpoint (sec) | 15 | 30 | 45 | 60 | 90 | 120 | 180 | 240 | 300 |
| Tumor | 0.566 | 0.570 | 0.561 | 0.555 | 0.557 | 0.548 | 0.547 | 0.542 | 0.533 |
| Muscle | 1.133 | 1.155 | 1.098 | 1.155 | 1.132 | 1.139 | 1.146 | 1.133 | 1.123 |
| Tumor/Muscle ratio | 0.500 | 0.494 | 0.511 | 0.480 | 0.492 | 0.481 | 0.477 | 0.479 | 0.475 |

| Frame | 9 | 10 | 11 | 12 | 13 | 14 | 15 |
|---|---|---|---|---|---|---|---|
| Duration (sec) | 60 | 180 | 180 | 180 | 300 | 300 | 300 |
| Midpoint (sec) | 360 | 540 | 720 | 900 | 1200 | 1500 | 1800 |
| Tumor | 0.525 | 0.508 | 0.499 | 0.484 | 0.474 | 0.477 | 0.497 |
| Muscle | 1.123 | 1.111 | 1.078 | 1.056 | 1.045 | 1.051 | 1.090 |
| Tumor/Muscle ratio | 0.468 | 0.458 | 0.463 | 0.459 | 0.454 | 0.454 | 0.456 |

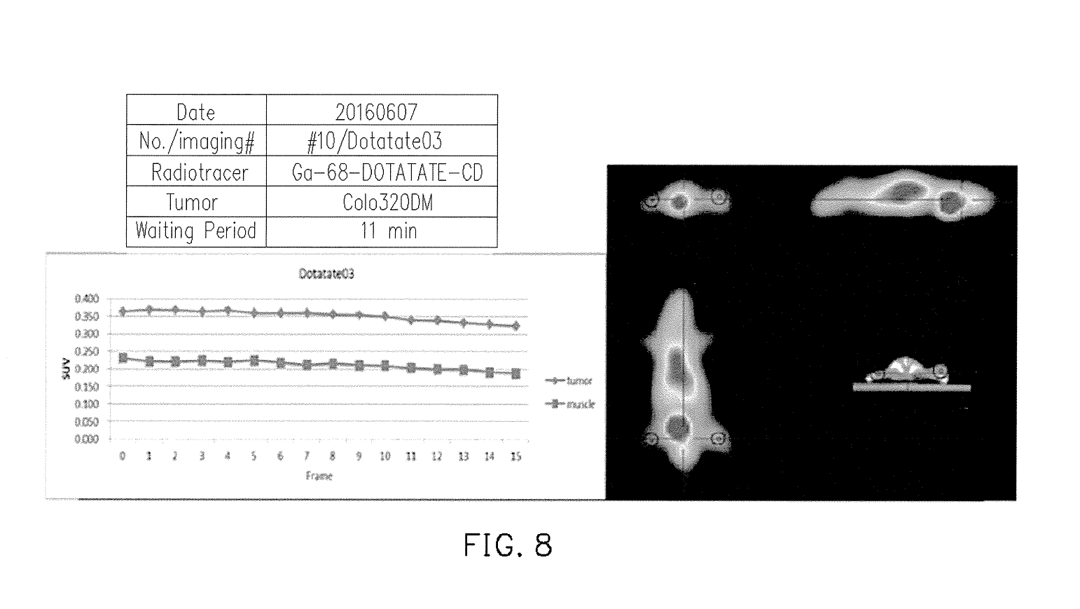

FIG. 8 is a PET/CT image of a human colorectal-tumor bearing mouse administered with ⁶⁸Ga-DOTATATE/CD according to Example 2 of the invention. The average tumor/muscle (T/M) count density ratios for ⁶⁸Ga-DOTATATE/CD in colorectal tumor models were calculated and presented in Table 5. Results from FIG. 8 and Table 5 indicated that the average tumor/muscle (T/M) count density ratios for ⁶⁸Ga-DOTATATE/CD in colorectal tumor-bearing mice were approximately in the range of 1.5 to 1.7. The results revealed that ⁶⁸Ga-DOTATATE/CD had a much higher tumor uptake than muscle as compared to ¹⁸F-FDG (T/M=0.8˜0.9) in colorectal tumor models, suggesting that ⁶⁸Ga-DOTATATE/CD is more sensitive than FDG in neuroendocrine tumor detection.

TABLE 5
Tumor/muscle (T/M) count density ratios for ⁶⁸Ga-DOTATATE/CD in colorectal tumor models

| Frame | 0 | 1 | 2 | 3 | 4 | 5 | 6 | 7 | 8 |
|---|---|---|---|---|---|---|---|---|---|
| Duration (sec) | 15 | 15 | 15 | 15 | 30 | 30 | 60 | 60 | 60 |
| Midpoint (sec) | 15 | 30 | 45 | 60 | 90 | 120 | 180 | 240 | 300 |
| Tumor | 0.364 | 0.369 | 0.367 | 0.364 | 0.367 | 0.359 | 0.359 | 0.359 | 0.355 |
| Muscle | 0.231 | 0.221 | 0.220 | 0.224 | 0.219 | 0.225 | 0.217 | 0.212 | 0.216 |
| Tumor/Muscle ratio | 1.571 | 1670 | 1.669 | 1.626 | 1.672 | 1.594 | 1.650 | 1.692 | 1.646 |

| Frame | 9 | 10 | 11 | 12 | 13 | 14 | 15 |
|---|---|---|---|---|---|---|---|
| Duration (sec) | 60 | 180 | 180 | 180 | 300 | 300 | 300 |
| Midpoint (sec) | 360 | 540 | 720 | 900 | 1200 | 1500 | 1800 |
| Tumor | 0.354 | 0.350 | 0.339 | 0.338 | 0.332 | 0.327 | 0.322 |
| Muscle | 0.211 | 0.210 | 0.203 | 0.200 | 0.198 | 0.191 | 0.188 |
| Tumor/Muscle ratio | 1.679 | 1.672 | 1.672 | 1.690 | 1.676 | 1.712 | 1.716 |

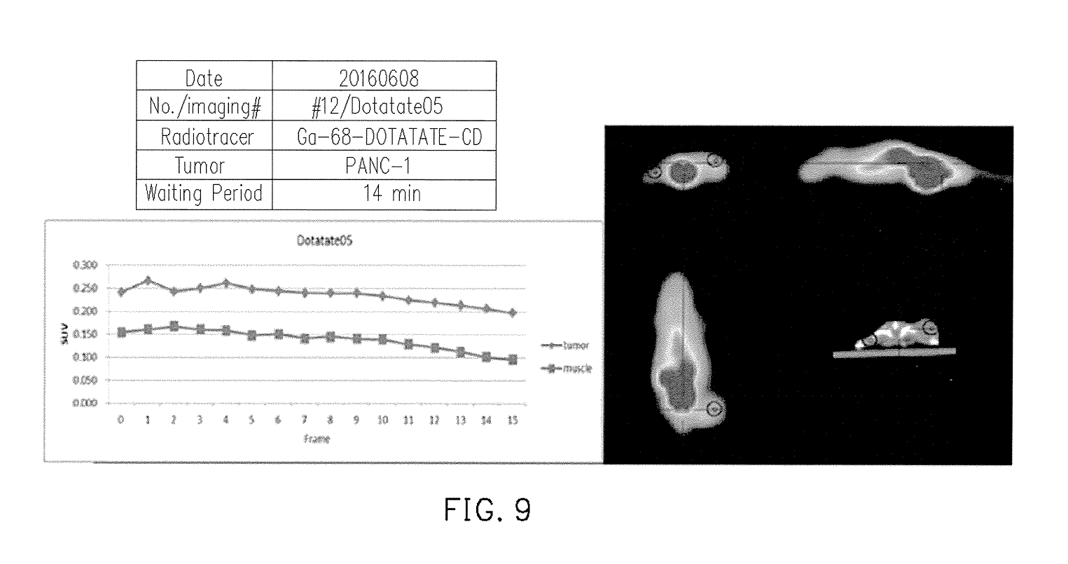

FIG. 9 is a PET/CT image of a human pancreatic-tumorbearing mouse administered with⁶⁸Ga-DOTATATE/CD according to Example 2 of the invention. The average tumor/muscle (T/M) count density ratios for ⁶⁸Ga-DOTATATE/CD in pancreatic tumor models were calculated and presented in Table 6. Results from FIG. 9 and Table 6 indicated that the average tumor/muscle (T/M) count density ratios for ⁶⁸Ga-DOTATATE/CD in pancreatic tumor-bearing mice were approximately in the range of 1.5 to 2.0. The results revealed that ⁶⁸Ga-DOTATATE/CD had a much higher tumor uptake than muscle as compared to ¹⁸F-FDG (T/M=0.8˜0.9) in pancreatic tumor models, proving again, that ⁶⁸Ga-DOTATATE/CD is more sensitive than FDG in neuroendocrine tumor detection.

TABLE 6
Tumor/muscle (T/M) count density ratios for ⁶⁸Ga-DOTATATE/CD in pancreatic tumor models

| Frame | 0 | 1 | 2 | 3 | 4 | 5 | 6 | 7 | 8 |
|---|---|---|---|---|---|---|---|---|---|
| Duration (sec) | 15 | 15 | 15 | 15 | 30 | 30 | 60 | 60 | 60 |
| Midpoint (sec) | 15 | 30 | 45 | 60 | 90 | 120 | 180 | 240 | 300 |
| Tumor | 0.241 | 0.266 | 0.242 | 0.250 | 0.261 | 0.247 | 0.244 | 0.240 | 0.239 |
| Muscle | 0.154 | 0.160 | 0.167 | 0.160 | 0.158 | 0.148 | 0.150 | 0.141 | 0.145 |
| Tumor/Muscle ratio | 1.561 | 1.660 | 1.454 | 1.561 | 1.647 | 1.672 | 1.622 | 1.703 | 1.651 |

| Frame | 9 | 10 | 11 | 12 | 13 | 14 | 15 |
|---|---|---|---|---|---|---|---|
| Duration (sec) | 60 | 180 | 180 | 180 | 300 | 300 | 300 |
| Midpoint (sec) | 360 | 540 | 720 | 900 | 1200 | 1500 | 1800 |
| Tumor | 0.239 | 0.233 | 0.224 | 0.219 | 0.213 | 0.206 | 0.197 |
| Muscle | 0.141 | 0.139 | 0.128 | 0.121 | 0.112 | 0.101 | 0.095 |
| Tumor/Muscle ratio | 1.699 | 1.675 | 1.742 | 1.803 | 1.898 | 2.040 | 2.062 |

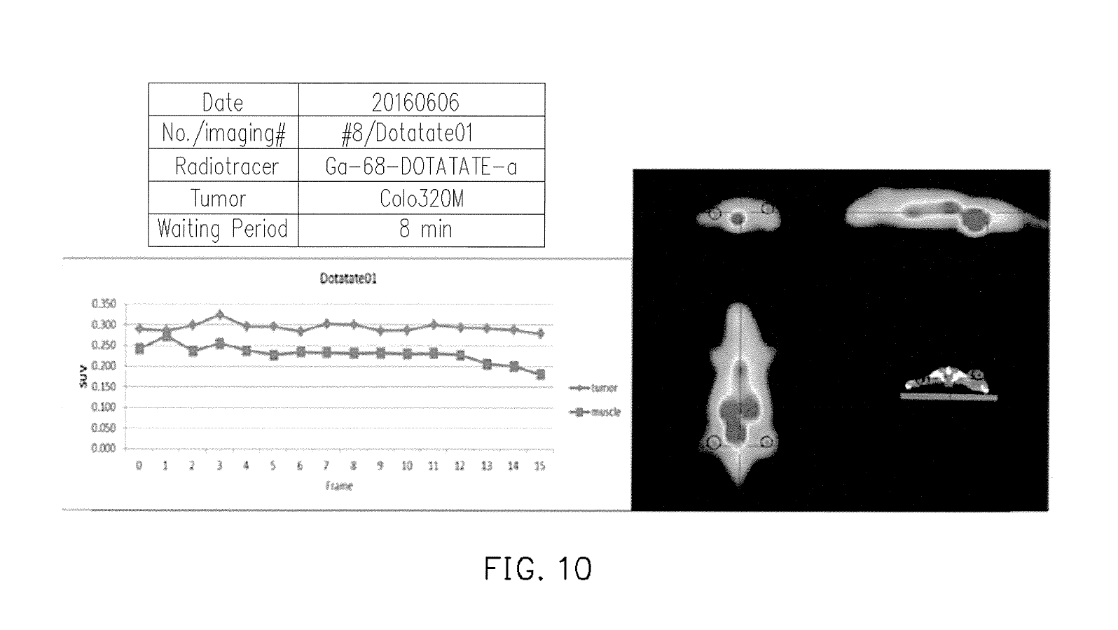

FIG. 10 is a PET/CT image of a human colorectal-tumor bearing mouse administered with ⁶⁸Ga-DOTATATE according to Example 2 of the invention. The average tumor/muscle (T/M) count density ratios for ⁶⁸Ga-DOTATATE in colorectal tumor models were calculated and presented in Table 7. Results from FIG. 10 and Table 7 indicated that the average tumor/ muscle (T/M) count density ratios for ⁶⁸Ga-DOTATATE in colorectal tumor-bearing mice were approximately in the range of 1.2 to 1.55. The results revealed that DOTATATE alone had a much higher tumor uptake than muscle as compared to ¹⁸F-FDG. However, DOTATATE (TM=1.2 to 1.55) showed slightly lower average tumor/muscle count density ratios as compared to DOTATATE/CD (TM=1.5 to 1.7). These results suggest that ⁶⁸Ga-DOTATATE/CD showed equal or better image findings than ⁶⁸Ga-DOTATATE. Hence, the presence of the transchelator (beta-cyclodextrin) can be used to provide better sensitivity in neuroendocrine tumor detection.

TABLE 7
Tumor/muscle (T/M) count density ratios for ⁶⁸Ga-DOTATATE in colorectal tumor models

| Frame | 0 | 1 | 2 | 3 | 4 | 5 | 6 | 7 | 8 |
|---|---|---|---|---|---|---|---|---|---|
| Duration (sec) | 15 | 15 | 15 | 15 | 30 | 30 | 60 | 60 | 60 |
| Midpoint (sec) | 15 | 30 | 45 | 60 | 90 | 120 | 180 | 240 | 300 |
| Tumor | 0.290 | 0.286 | 0.298 | 0.324 | 0.296 | 0.296 | 0.284 | 0.303 | 0.300 |
| Muscle | 0.242 | 0.273 | 0.237 | 0.255 | 0.238 | 0.227 | 0.235 | 0.233 | 0.231 |
| Tumor/Muscle ratio | 1.197 | 1.049 | 1.259 | 1.269 | 1.240 | 1.302 | 1.210 | 1.300 | 1.298 |

| Frame | 9 | 10 | 11 | 12 | 13 | 14 | 15 |
|---|---|---|---|---|---|---|---|
| Duration (sec) | 60 | 180 | 180 | 180 | 300 | 300 | 300 |
| Midpoint (sec) | 360 | 540 | 720 | 900 | 1200 | 1500 | 1800 |
| Tumor | 0.286 | 0.287 | 0.300 | 0.294 | 0.292 | 0.288 | 0.279 |
| Muscle | 0.232 | 0.230 | 0.231 | 0.227 | 0.206 | 0.200 | 0.180 |
| Tumor/Muscle ratio | 1.234 | 1.249 | 1.298 | 1.293 | 1.419 | 1.442 | 1.548 |

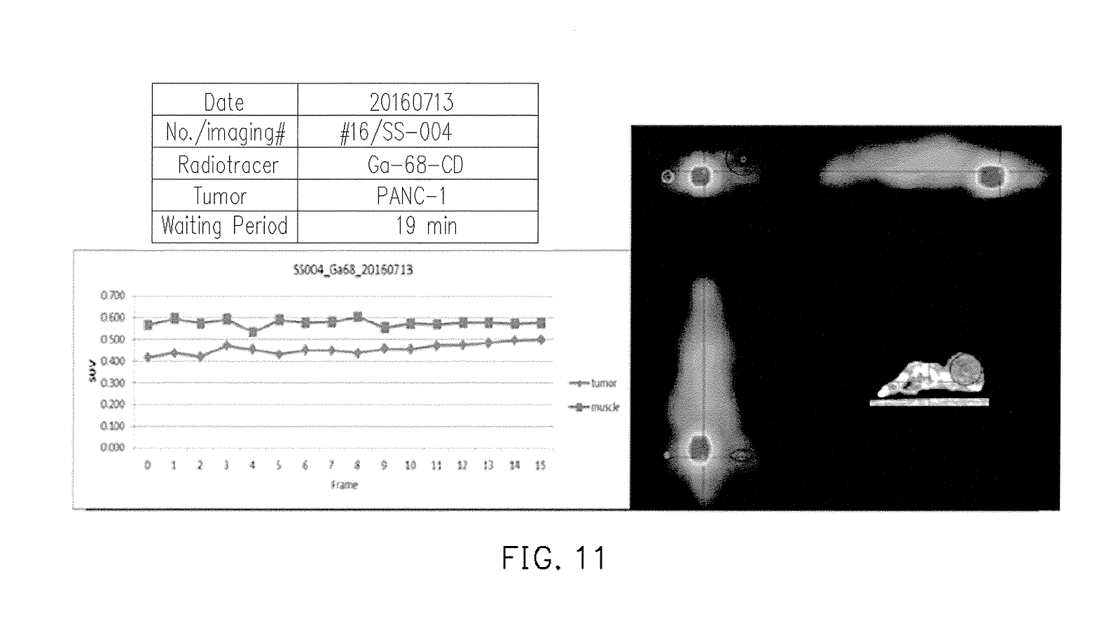

FIG. 11 is a PET/CT image of a human pancreatic-tumor bearing mouse administered with ⁶⁸Ga-CD according to Example 2 of the invention. The average tumor/muscle (T/M) count density ratios for ⁶⁸Ga-CD in pancreatic tumor models were calculated and presented in Table 8. Results from FIG. 11 and Table 8 indicated that the average tumor/muscle (T/M) count density ratios for ⁶⁸Ga-CD in pancreatic tumor-bearing mice were approximately in the range of 0.73-0.86. The results revealed that cyclodextrin alone had slightly higher tumor/muscle density ratios as compared to FDG. However, the tumor/muscle density ratios of cyclodextrin alone are not comparable to that of ⁶⁸Ga-DOTATATE or ⁶⁸Ga-DOTATATE/CD. These results suggest that the higher tumor uptake in ⁶⁸Ga-DOTATATE/CD is not attributed to ⁶⁸Ga-DOTATATE or cyclodextrin alone, instead, the combination of ⁶⁸Ga-DOTATATE and cyclodextrin is important for achieving higher sensitivity in neuroendocrine tumor detection. As such, cyclodextrin is a useful transchelator in DOTATATE imaging and theranostic applications.

TABLE 8
Tumor/muscle (T/M) count density ratios for ⁶⁸Ga-CD in pancreatic tumor models

| Frame | 0 | 1 | 2 | 3 | 4 | 5 | 6 | 7 | 8 |
|---|---|---|---|---|---|---|---|---|---|
| Duration (sec) | 15 | 15 | 15 | 15 | 30 | 30 | 60 | 60 | 60 |
| Midpoint (sec) | 15 | 30 | 45 | 60 | 90 | 120 | 180 | 240 | 300 |
| Tumor | 0.418 | 0.437 | 0.421 | 0.470 | 0.453 | 0.432 | 0.450 | 0.449 | 0.438 |
| Muscle | 0.565 | 0.594 | 0.574 | 0.592 | 0.534 | 0.589 | 0.578 | 0.581 | 0.604 |
| Tumor/Muscle ratio | 0.739 | 0.736 | 0.733 | 0.795 | 0.848 | 0.733 | 0.779 | 0.773 | 0.724 |

| Frame | 9 | 10 | 11 | 12 | 13 | 14 | 15 |
|---|---|---|---|---|---|---|---|
| Duration (sec) | 60 | 180 | 180 | 180 | 300 | 300 | 300 |
| Midpoint (sec) | 360 | 540 | 720 | 900 | 1200 | 1500 | 1800 |
| Tumor | 0.456 | 0.454 | 0.472 | 0.473 | 0.484 | 0.495 | 0.498 |
| Muscle | 0.554 | 0.574 | 0.569 | 0.578 | 0.577 | 0.572 | 0.576 |
| Tumor/Muscle ratio | 0.822 | 0.792 | 0.830 | 0.818 | 0.838 | 0.866 | 0.865 |

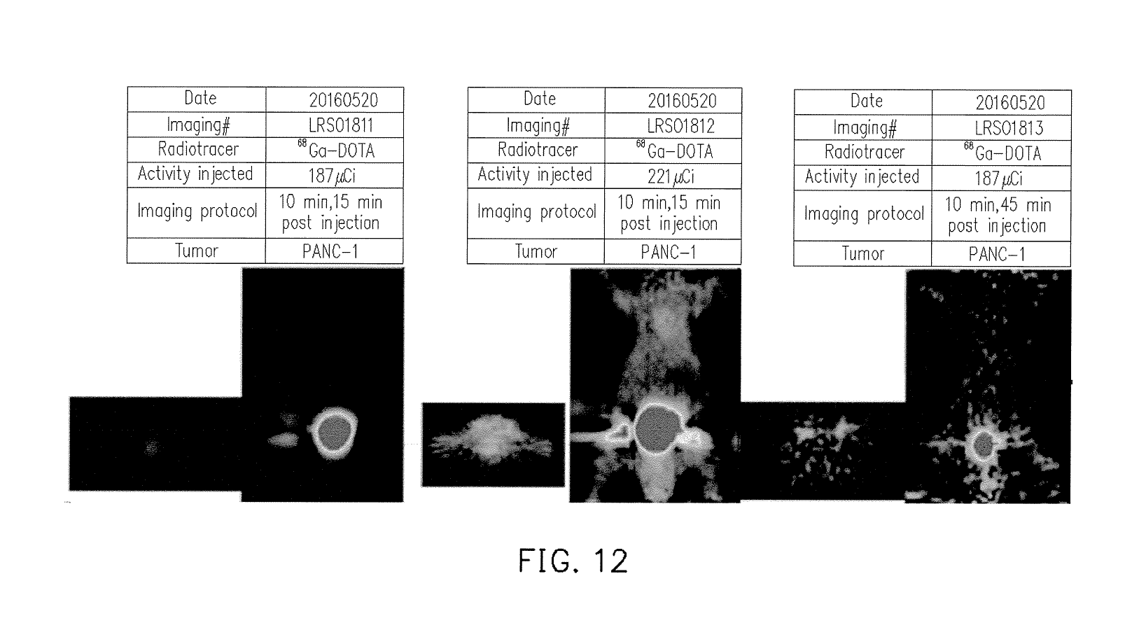

FIG. 12 is a PET/CT image of a human pancreatic-tumor bearing mouse administered with ⁶⁸Ga-DOTA according to Example 2 of the invention. As shown in FIG. 12, ⁶⁸Ga-DOTA could not be used to visualize neuroendocrine tumors due to its fast clearance and high bladder uptake. The results prove that DOTATATE is the more preferable choice for the chelator-somatostatin receptor ligand and for achieving higher sensitivity in neuroendocrine tumor detection.

In summary, the pharmaceutical formulation of the present invention includes a chelator-somatostatin receptor ligand and a transchelator, wherein the transchelator is capable of capturing free metal source or radionuclide source that is not conjugated to the chelator-somatostatin receptor ligand. Since the transchelator is capable of capturing all of the free/unbound metal source or radionuclide source, thus column purification steps can be avoided, and the preparation of radiolabeled somatostatin analogues could be made more efficient, and be feasible for imaging of SSTR pathway-activated systems in cancers and neurological diseases. In particular, ⁶⁸Ga-DOTATATE in combination with beta-cyclodextrin as a transchelator was found to achieve optimal sensitivity in neuroendocrine tumor detection.

Currently, marketed drug may not generate sufficient return to justify the investment due to complex manufacturing of radiopharmaceuticals. The new formulation provided in the present invention benefits to the patients due to fast cGMP preparation, and the ability to produce high yield products within minutes. The advantages of this new formulation can be summarized as follows. Firstly, the whole processes are operated in a closed system that can provide consistency and reduce the environmental radiation. Secondly, the process time is about 10 min with purity of greater than 95% which is sufficient to meet the requirements of the specifications for Ga-68-DOTATATE PET in nuclear medicine applications. Lastly, the temperature and variation of radiation dose can be monitored during the process which follows the compliance with the regulation of cGMP.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A pharmaceutical formulation, comprising:
a chelator-somatostatin receptor ligand, wherein the chelator-somatostatin receptor ligand is conjugated with a metal source or a radionuclide source; and
a transchelator, capable of capturing free metal source or radionuclide source that is not conjugated to the chelator-somatostatin receptor ligand, wherein a pH of the chelator-somatostatin receptor ligand conjugated with the metal source or the radionuclide source is between 4 to 5.

2. The pharmaceutical formulation according to claim 1, wherein the chelator-somatostatin receptor ligand is used as an active ingredient.

3. The pharmaceutical formulation according to claim 1, wherein the radionuclide source is selected from the group of metal ions including ^{99m}Tc, ^{117m}Sn, ¹⁷⁷Lu, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹n, ¹⁸³Gd, ⁵⁹Fe, ²²⁵Ac, ²²³Ra, ²¹²Bi, ²¹¹At, ⁴⁵Ti, ⁶⁰Cu, ⁶¹Cu, ⁶⁷Cu, ⁶⁴Cu and ⁶²Cu, and wherein the metal source is a non-radioactive metal such as ¹⁸⁷Re, ⁶⁹Ga, ¹⁵³Pt.

4. The pharmaceutical formulation according to claim 1, wherein the chelator-somatostatin receptor ligand is octreotide ligands selected from DOTA-TOC, DOTATATE, DOTA-NOC or DTPAOC.

5. The pharmaceutical formulation according to claim 1, wherein the transchelator is citrate, mannitol, alpha-cyclodextrin, beta-cyclodextrin, gamma-cyclodextrin, hydroxypropyl cyclodextrin, glucose, glucosamine, gluconate, glucarate, glucoheptonate.

6. The pharmaceutical formulation according to claim 1, wherein the chelator-somatostatin receptor ligand is DOTATATE and the transchelator is beta-cyclodextrin, wherein a usage amount of the beta-cyclodextrin is in a range from 1 mg to 100 mg for every 100 μg of the DOTATATE, and the pH of the chelator-somatostatin receptor ligand conjugated with the metal source or the radionuclide source is between 4 to 5.

7. A method of preparing a pharmaceutical formulation, comprising the following steps:
reacting a chelator-somatostatin receptor ligand with a metal source or a radionuclide source so that the metal source or the radionuclide source is conjugated to the chelator-somatostatin receptor ligand, wherein no column purification step is performed after reacting the chelator-somatostatin receptor ligand with the metal source or the radionuclide source; and
mixing the chelator-somatostatin receptor ligand with a transchelator.

8. The method of preparing the pharmaceutical formulation according to claim 7, wherein the pharmaceutical formulation is prepared into one of the following forms for administration: tablets, capsules, powders, dispersible granules, cachets and suppositories, sustained release and delayed release formulations, liquid dosage forms, solutions, suspensions and emulsions, injectable formulations, solutions or sprays for intranasal, buccal or sublingual administration, aerosol preparations suitable for inhalation, transdermal formulations, creams, lotions, aerosols and/or emulsions and transdermal patches.

9. The method of preparing the pharmaceutical formulation according to claim 8, wherein the pharmaceutical formulation is administered intravenously.

10. The method of preparing the pharmaceutical formulation according to claim 7, wherein the radionuclide source is selected from the group of metal ions including ^{99m}Tc, ^{117m}Sn, ¹⁷⁷Lu, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²²³Ra, ²¹²Bi, ²¹¹At, ⁴⁵Ti, ⁶⁰Cu, ⁶¹Cu, ⁶⁷Cu, ⁶⁴Cu and ⁶²Cu, and wherein the metal source is a non-radioactive metal such as ¹⁸⁷Re, ⁶⁹Ga, ¹⁵³Pt.

11. The method of preparing the pharmaceutical formulation according to claim 7, wherein the chelator-somatostatin receptor ligand is octreotide ligands selected from DOTA-TOC, DOTATATE, DOTA-NOC or DTPAOC.

12. The method of preparing the pharmaceutical formulation according to claim 7, wherein the transchelator is citrate, mannitol, alpha-cyclodextrin, beta-cyclodextrin, gamma-cyclodextrin, hydroxypropyl cyclodextrin, glucose, glucosamine, gluconate, glucarate, glucoheptonate.

13. The method of preparing the pharmaceutical formulation according to claim 7, wherein the chelator-somatostatin receptor ligand is DOTATATE and the transchelator is beta-cyclodextrin, wherein based on 100 μg of DOTATATE, a usage amount of the beta-cyclodextrin is in a range from 1 mg to 100 mg, and a pH of the pharmaceutical formulation is between 4 to 5.

14. A method of imaging neuroendocrine tumor in a patient using nuclear imaging, wherein the method comprises administering to the patient an effective amount of the pharmaceutical formulation according to claim 1, wherein the chelator-somatostatin receptor ligand conjugated with the metal source or the radionuclide source is ⁶⁸Ga-DOTATATE or ^{99m}Tc-DOTATATE; and the transchelator is beta-cyclodextrin.

15. The method of imaging neuroendocrine tumor according to claim 14, wherein the neuroendocrine tumor is selected from the group consisting of brain tumor, breast tumor, prostate tumor, colon tumor, lung tumor, liver tumor, pancreas tumor, gastric tumor, lymphoma, uterine tumor, cervical tumor, thyroid tumor and melanoma.

16. The method of imaging neuroendocrine tumor according to claim 14, wherein the nuclear imaging used is positron emission tomography (PET) or single photon emission computed tomography (SPECT).

17. A method of imaging somatostatin receptor system in a patient with neurological diseases and psychiatric disorder using nuclear imaging, wherein the method comprises administering to the patient an effective amount of the pharmaceutical formulation according to claim 1.

18. The method of imaging somatostatin receptor system according to claim 17, wherein the neurological diseases and the psychiatric disorder are selected from the group consisting of Alzheimer, Huntington's disease, Parkinson, Epilepsy, Amyotrophic lateral sclerosis (ALS), Posttraumatic stress disorder (PTSD), Attention deficit hyperactivity disorder (ADHD), dementia, mood disorders and psychic symptoms.

19. The method of imaging somatostatin receptor system according to claim 17, wherein the nuclear imaging used is positron emission tomography (PET) or single photon emission computed tomography (SPECT).
